(19) United States
(12) Patent Application Publication
(10) Pub. No.: US 2010/0106245 A1
(43) Pub. Date: Apr. 29, 2010

(54) LOW PCO HAPTICS FOR INTRAOCULAR LENS

(75) Inventors: Michiel Christiaan Rombach, Breda (NL); Aleksey Nikolaevich Simonov, Delft (NL)

Correspondence Address: Barnes & Thornburg LLP, Atlanta Financial Center, 3343 Peachtree Road N.E., Suite 1150, Atlanta, GA, 30326-1428, US

(73) Assignee: AKKOLENS INTERNATIONAL B.V., BA Breda (NL)

(21) Appl. No.: 12/523,758

(22) PCT Filed: Jan. 28, 2008
(86) PCT No.: PCT/NL08/50049

§ 371 Date: Dec. 7, 2009

(30) Foreign Application Priority Data
Jan. 26, 2007 (EP) 07101267.8

(51) Int. Cl. A61F 2/16

(52) U.S. Cl. 623/6.39

(57) ABSTRACT

An intraocular lens comprising a central optical element (5) and at least two haptics (1) positioned in a plane perpendicular to the optical axis of the eye, wherein at least one haptic has a substantially Ω-shaped structure adapted to be compressed in a direction perpendicular to the optical axis, and wherein the optical surfaces of the central optical element are smooth over their full areas. Two Ω-shaped spring-like haptics (6) with different flexibility may be combined with these features resulting in an accommodating lens when the haptics are mechanically coupled to a structure of the eye subject to movements like the sulcus or the capsular bag.

### PRIORITY CLAIM OR CROSS REFERENCE TO RELATED APPLICATIONS

This patent application is a U.S. National Phase of International Patent Application No. PCT/NL2008/050049, filed Jan. 28, 2008, which claims priority to European Patent Application No. 07101267.8, filed Jan. 26, 2007, the disclosures of which are incorporated herein by reference in their entirety.

### FIELD

The present disclosure relates to an intraocular lens having a central optical element and at least two haptics.

### BACKGROUND

Intraocular lenses (hereinafter referred to as “IOLs”) are generally used to treat the eyes of patients in which cataracts cloud the natural lens of the eye. Untreated eyes gradually become blind, but cataract surgery can restore clear vision. During cataract surgery, the eye surgeon removes the clouded natural lens from the capsular bag though a hole, a capsulorrhexis, in the capsular bag, the lens' natural cavity and holder, and implants a transparent polymer artificial IOL to replace the natural lens. Cataract surgery is a standard surgical procedure which is carried out approximately 30 million times each year worldwide.

Such standard transparent polymer monofocal and multifocal artificial IOLs are comprised of at least one optical element, hereinafter referred to as “optics”, and positioning/attachment components, known as “haptics”, to position these optics in the eye. The optics are generally 5-6 mm in diameter and the haptics are fastening components attached to the rim of the optics to position and fasten the optics to, generally, the rim of the capsular bag and into the eye.

The optics determine the quality of vision, but the haptics are also of crucial importance for the proper long term functioning of the IOL. The present disclosure provides new designs and new properties of IOL haptics, including haptics arrangements which allow a single optic to shift perpendicular (transversely) to the optical axis.

Firstly, the haptics should be made of biocompatible materials, generally the same material as the optics, e.g., PMMA, acrylate or silicone, but not necessarily the same material. The haptics can also be made of different materials of which optics are not made. These materials include polyamide, polypropylene, nylon, and the like, and even various metals which are glued or otherwise firmly attached to the optics components of the IOL.

Secondly, modern IOLs are all foldable or rollable to fit the cartridge of an IOL injector. Injection of an IOL simplifies surgery, allowing for smaller incisions in the eye which can be so small that no stitching is required at the end of surgery. Haptics should, therefore, also be foldable or rollable and not hamper such injectability of the IOL.

Thirdly, the haptics must have such a design that the haptics position the IOL into the eye and provide long term stability, centration and prevention of tilt of the optics. IOL malpositioning can range from IOL decentration to even luxation into the posterior segment of the eye. Subluxated IOLs involve such extreme decentration that the IOL optic covers only a small fraction of the pupillary space. Luxation involves total dislocation of the IOL into the posterior segment. Decentration of an IOL can be the result of the original surgical placement of the lens or decentration may develop in the postoperative period, e.g., due to severe capsular bag contraction. It is known that haptics can affect capsular bag shrinkage, but the mechanisms of this effect are not well known. Decentration of clinical insignificance occurs in at least 25% of cases, clinically significant decentration occurs in about 3% of the cases and the frequency of IOL dislocation ranges from 0.2-1.8%. Proper haptics and proper haptics fit in the eye can prevent the majority of such dislocations.

Fourthly, haptics should be designed such that the incidence of Post Cataract Opafication (hereinafter referred to as “PCO”) of the capsular bag and the occurrence of secondary cataracts are minimized. PCO occurs generally and in approximately 10-20% of the eyes implanted with an IOL. PCO can be treated with a YAG-laser treatment at a later stage which is a standard treatment for PCO. However, such additional surgery carries a) additional medical risk and b) additional financial costs, and prevention of PCO is a major issue for surgeons and their patients.

Fifthly, the design and assembly of haptics should fit a manufacturing production procedure of the IOL. Ease of manufacturing becomes an ever increasingly important aspect of IOL design because of falling IOL prices worldwide. For example, 3-piece IOLs (e.g., acrylate optics with two glued-in PMMA haptics) are popular but are expensive to produce compared to silicone lenses which can be molded in mass.

Sixthly, haptics can be designed such that a shift of the optics occurs during the accommodation process of the eye. Such shift is generally a shift of at least one monofocal optics along the optical axis which results bringing objects closer to the eye in focus. However, certain optics achieve a focusing effect by a shift perpendicular to the optical axis, e.g., lenses made of a pair of cubic surfaces, resulting in optically near perfect accommodation, and single progressive lenses.

Haptics designs are manifold and fall into the following broad categories, of which only few examples are given below to illustrate the various increasingly complex designs. Such designs can be haptics as open loops, mostly C-loops (e.g., as disclosed in International Patent Publication Nos. WO 2006/023386 and WO 2005/082287), closed loops/plate haptics (e.g., as disclosed in International Patent Publication No. WO 2006/124274), haptics which form a mix of these designs (e.g., as disclosed in U.S. Patent Publication No. 2006/276892 and European Patent Nos. 1658828 and 1,502,561), haptics with additional ring-like support components (e.g., as disclosed in Canadian Patent Application No. 2,530,033), haptics with a T-shaped structures (e.g., as disclosed in European Patent No. 1627614 and Japanese Patent Application No. 2005161075) or variations thereon (e.g., as disclosed in U.S. Patent Publication No. 2005/246017; European Patent No. 1543799; and U.S. Patent Publication No. 2005/107875), haptics with more complex structures (e.g., a spring-structure as disclosed in U.S. Pat. No. 6,986,787 for one single lens or spring-structures for multiple lens systems as disclosed in International Patent Publication No. WO 2005/065591, and haptics with multiple complex components as disclosed in U.S. Patent Publication Nos. 2005/096741 and 2005/113914). These springs function move one lens or multiple lenses along the optical axis, generally with the intention to provide the eye with a level of accommodation, the haptics and optics being driven by the natural ciliary muscle of the eye. Also, adjustable haptics have been described for use with IOLs (e.g., as disclosed in International Patent Publication No. WO 2005/000551). This listing has no other intention than to provide a few characteristic examples of haptics designs from an exhaustive list of existing patent literature.

Open loops are generally referred to as C-loop haptics in which the haptics form part of the total IOL construction and are manufactured from the same materials as the optics. Other, so called 3-piece IOLs, have C-loop haptics manufactured from a different material and attached to the optics by mostly precision drilling and subsequent gluing of C-loop haptics into the holes drilled in the optics components.

Closed loop haptics have a closed loop with one or more openings/holes intended for fluid exchange between the front part and the back part of the IOL. Closed loop haptics can also be plate haptics and be composed of single or multiple larger sturdy plates, with or without holes. These plate haptics are large plates extending from the optics often providing the IOL with a more or less rectangular shape. Posterior dislocation is a well-described complication of plate-haptic IOLs. It can occur after an opening in the posterior capsule, either intra-operatively or after a YAG capsulotomy occurs. There is a need for a small and continuous capsulorhexis as well as in-the-bag implantation of plate-haptic IOLs. This additional requirement on the surgeon can make this type of IOL less preferred.

Various haptics designs position and stabilize the IOL. However, the incidence of post-surgery cataract varies significantly with designs. Clearly, having a biocompatible material for the haptic is not sufficient to prevent PCO and secondary cataract formation. Also the amount and direction of the forces exuded by the haptics on the capsular bag and other components of the eye play a role in PCO formation.

Certain Ω-shaped spring-like haptics which function to shift optical elements perpendicular to the optical axis have been described in International Patent Publication No. WO 2005/084587; Netherlands Patent Application No. 1028496; International Patent Publication No. WO 2006/118452; and European Patent No. 1720489. Firstly, the behaviour of the 1)-shaped spring-like haptics was simulated in advanced Finite Element Models (hereinafter referred to as “FEM”), optics and haptics were manufactured by precision lathing and milling and the IOLs constructions were tested in medical trials. Accommodating IOLs with two optical elements and such spring-like haptics were tested in medical trials to have the IOL focused by shifting optical elements by the natural system of the ciliary muscle of the eye. These spring-like haptics resulted in nearly negligible incidence of PCO and secondary cataract formation in the eye. These Ω-shaped spring-like haptics are, therefore, claimed for use with non-accommodating, i.e., monofocal, IOL optics and multifocal IOL optics which have at least one fixed optical focal point.

Additionally, movement of the optics can be achieved by combining at least one flexible haptic with, on the opposite site of the optics, at least one rigid Ω-shaped spring-like haptic. Such construction can shift a smooth or discrete, bifocal, multifocal or progressive optics perpendicular to the optical axis of the eye and focal change of the eye can be achieved. The movement can be driven by either the ciliary muscle directly via the natural accommodation process, with the construction, for example, inside the capsular bag or in front of the capsular bag. Alternatively, such construction can be positioned in the sulcus of the eye, in front of the capsular bag. The sulcus also decreases its diameter in parallel with the ciliary muscle when the eye accommodates.

The aim with these Ω-shaped spring-like haptics is to have a stretching force on the capsular bag sufficient to stretch the capsular bag towards the ciliary mass/sulcus of the eye, but with a resulting force on the ciliary mass/sulcus which is minimal and as close to a zero-force as can be achieved. With application of such Ω-shaped spring-like haptics and the calibration of forces, an unusual low incidence of PCO and secondary cataract formation occurs due to as little stimulation as possible of the pressure sensitive epithelial cells which are responsible for PCO and likely also play a role in triggering secondary cataract formation.

### SUMMARY

The present disclosure describes several exemplary embodiments of the present invention.

One aspect of the present disclosure provides an intraocular lens, comprising a) a central optical element having optical surfaces which are smooth over their full areas; and b) at least two haptics positioned in a plane perpendicular to the optical axis of the eye, wherein at least one of the haptics has a substantially Ω-shaped structure adapted to be compressed in a direction perpendicular to the optical axis.

These features combine the aspects of the Ω-shaped haptics disclosed hereinabove with those of a lens having two smooth surfaces, like a progressive lens.

In one exemplary embodiment, the haptic, in particular, the Ω-shaped part or loop of the haptic, is positioned in a plane perpendicular to the optical axis of the eye. The present disclosure provides Ω-shaped spring-like haptics in combination with, but not restricted to, standard fixed monofocal lenses, fixed multifocal lenses, rotational asymmetrical multifocal lenses and progressive optics, including progressive optics with azimuthal progression. These progressive lenses with azimuthal progression are lenses wherein the optical strength increases in the vertical direction, preferably between two haptics. This provides a lens which is not only smooth on both optical surfaces but also has progressive optical properties. Two Ω-shaped spring-like haptics with different flexibility may be combined with these features, resulting in an accommodating lens when the haptics are mechanically coupled to a structure of the eye subject to movements like the sulcus or the capsular bag. The haptics should be positioned in the line according to which the optical properties of the lens progress so that the movements of the lens are parallel to the direction of optical progression. Such Ω-shaped spring-like haptics have at least one flat Ω-shaped spring-like structure which acts like a spring and as attachment component with the spring-like action in precisely the same plane as the plane of the optics which are positioned in a plane perpendicular to the optical axis of the eye and which spring/attachment combination functions like a haptic to position, hold, stabilize, and, if designed so, move the IOL optics perpendicular to the optical axis of the eye.

Secondly, such Ω-shaped spring-like haptics have a spring with a force such that the capsular bag is stretched, fully stretched or stretched to a predetermined degree of stretching, but the stretching occurs to such a degree that the force depressing the ciliary mass/sulcus or the force resulting to the sulcus is minimized, wherein the force is preferably low and as close to a zero-force as possible. This exertion of force is crucial to proper functioning of the haptics. Epithelial cells which cover transparent components of the eye are generally organized in one layer, and these cells have to be pressure sensitive to maintain this one layer arrangement. Forces exerted the layer in a longitudinal direction will affect cell division and cellular arrangement. Precise distribution of forces will prevent the trigger of epithelial cells in repeated cell division leading to PCO. The capsular bag should be stretched sufficiently to prevent shrinkage, but stretching force should be limited to prevent PCO. Also, it is highly likely that dividing epithelial cells also trigger formation of secondary cataracts or play a major role in such formation. Reduction of capsular bag shrinkage, PCO and secondary cataract formation is thus obtained.

Therefore, one exemplary embodiment provides that the elastic force of the haptic increases the diameter in the direction of the haptic up to 5%.

In another exemplary embodiment, one flat Ω-shaped spring-like haptic is applied in combination with a rigid, non-elastic haptic with no or low spring-like action of any shape, but generally with a similar radius to the flat Ω-shaped spring-like haptic at the opposite side of the optics. Clearly, special consideration must be given to alignment in the eye, especially with respect to the central part of the optics in relation to the optical axis of the eye. This exemplary embodiment allows the eye to shift the optics perpendicular to the optical axis, which can result in an accommodative effect with the proper design of optics, e.g., multifocal designs, like lenses with progressive optical properties. According to a preferred exemplary embodiment, the optical strength of the lens increases in the direction between the two haptics. When one of the haptics has a flexibility different from the other haptic, contraction of a structure in the eye in which the lens is located will lead to a movement of the lens relative to the optical axis, allowing the positioning of parts of the lens having different optical properties in the optical axis and hence to accommodate the eye.

Also, two such Ω-shaped spring-like structures can be attached symmetrically to the optical component, or any number of such flat Ω-shaped spring-like haptics can be attached to the optical component, symmetrically or asymmetrically, in combination with any number of non-spring-like haptics of a different shape.

Alternatively, an uneven number or an even number of such flat Ω-shaped spring-like haptics can be distributed evenly along the rim of the optics of the intraocular lens with, depending on the size of the individual flat Ω-shaped spring-like haptics, the combination forming a circular flat spring-like structure.

Also, a mix of rigid, non-elastic haptics and flat Ω-shaped spring-like haptics can likewise be distributed along the rim of the optics. The rigid, non-elastic haptics will support stability but the rigid, non-elastic haptics should be positioned somewhat closer to the rim of the optics so as not to hamper the spring-like effects of the flat Ω-shaped spring-like haptics.

Such flat Ω-shaped spring-like haptics have a spring with a force such that the capsular bag is stretched, fully stretched or stretched to a predetermined degree of stretching but stretched to such a degree that the force depressing the ciliary mass or the force resulting to the sulcus is minimalized with the force being exerted to the ciliary mass/sulcus as close to a zero-force as possible.

A method for calibrating resulting forces described hereinabove includes measuring the diameter of the ciliary body (distance of “ciliary-mass-to-ciliary-mass”) or diameter of the sulcus (distance “sulcus-to-sulcus”) and providing the eye with an IOL which has a size such that the resulting forces will be in the order of magnitude as described hereinabove. Clearly, a proper diameter of the construction to fit the position in the eye is crucial for designs which move optics perpendicular to the optical axis. Improper diameter likely results in an anemmetrope eye. Such measurement of the diameter can be accomplished by modern UBM-ultrasound technology with great accuracy. Also, such measurements can be accomplished by penetration of the eye through the surgical incision by which the natural lens was removed by a small and flexible, e.g., polymer, strip or small ruler. The desired size can then be concluded from size markings on the strip or, alternatively, be estimated from the degree with which the strip bends after coming in contact with the ciliary mass/sulcus opposite the point of entry.

The flat Ω-shaped spring-like haptics and additional haptics of any other shape can be manufactured by modern IOL milling technology. Such manufacturing was shown for production batches in manufacturing of lenses disclosed in International Patent Publication No. WO 2005/084587. The flat Ω-shaped spring-like haptics in these designs are similar to the flat Ω-shaped spring-like haptics described in the present disclosure and hold their shape and spring-like action in different IOL grade materials even after extended periods of time with a profound reduction of PCO, capsular bag shrinkage and secondary cataract formation.

The optics, the flat Ω-shaped spring-like haptics and, if included in the design of the construction, additional otherwise shaped non-elastic haptics or other additional components to the construction can be manufactured from different materials with different mechanical and optical properties and assembled into a final construction after individual manufacturing. Alternatively, the final construction with two different materials can be manufactured in one production procedure by lathing and milling from modern “duo-materials”, i.e., material buttons for IOL manufacturing which consist of two different materials, generally with a core (e.g., a hydrophilic acrylate or hydrophobic acrylate) for optics manufacturing by lathing surrounded by a mantle (e.g., PMMA/perspex) for haptics manufacturing by subsequent milling around the central optics core following lathing of the optics.

Earlier designs of IOLs (such as those disclosed in International Patent Publication No. WO 2005/084587) with such flat Ω-shaped spring-like haptics consisted of two optical elements connected by the haptics. These IOLs are produced by lathing, milling and assembly by re-polymerization of a strip of the haptics. Such basically 3D constructions with two optics are difficult to produce by molding, and can only be produced by application of precision inserts. IOLs with only one optic can generally be molded. The flat Ω-shaped spring-like haptics disclosed herein can be produced by molding in combination with an IOL with a one-optic configuration from, for example, silicone materials.

The intraocular lens with flat Ω-shaped spring-like haptics can be combined with adapted, but further standard capsular rings, e.g., manufactured from PMMA, to further stabilize the design in the capsular bag. Clearly, the forces exerted by the rings should be calibrated as not to disturb the alignment of forces disclosed hereinabove.

### BRIEF DESCRIPTION OF THE DRAWINGS

Various aspects of the present disclosure are described hereinbelow with reference to the accompanying figures.

FIG. 1 shows one exemplary embodiment of an intraocular lens with a single flat Ω-shaped spring-like haptic with a rim;

FIG. 2 shows a second exemplary embodiment of an intraocular lens with a flat Ω-shaped spring-like haptic;

FIG. 3 shows a third exemplary embodiment of an intraocular lens having three flat Ω-shaped spring-like haptics arranged around the optics; and

FIG. 4 shows a fourth exemplary embodiment of an intraocular lens having four flat Ω-shaped spring-like haptics forming a circular spring-like ring around the optics.

### DETAILED DESCRIPTION

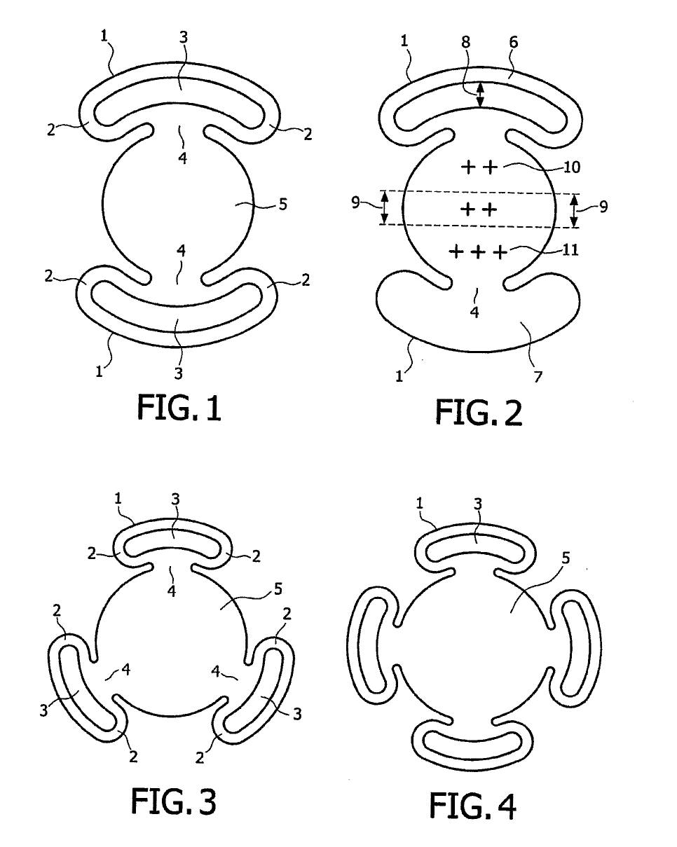

A first exemplary embodiment of an intraocular lens shown in FIG. 1 has one flat Ω-shaped spring-like haptic opposite a sturdy haptic. The optics are, for example, of a non-rotational symmetrical multifocal or progressive design to allow a change in accommodation status of the eye at shifting of the optics perpendicular to the optical axis of the eye. This design provides a reduction in PCO and changes in accommodative status of the eye. Such intraocular lens can be implanted in the capsular bag or, likely with adaptations, in the sulcus of the eye.

A second exemplary embodiment of an intraocular lens shown in FIG. 2 has two flat Ω-shaped spring-like haptics opposing each other. The optics are of a rotational symmetrical multifocal or monofocal design. This design provides a reduction in PCO.

It is possible to adapt the haptics to locate the lens in the capsular bag as is known per se. The advantages mentioned hereinabove will then become apparent. The lens, however, may also be positioned in other locations in the eye, for example, with the haptics positioned in the sulcus. The sulcus of the eye also executes movements related to the circular muscle of the eye and the sulcus can also be used as a structure to drive the lens. The sulcus allows a form locking connection with the haptics, firstly, designs with flanges extruding from the rim of the haptics which flanges have dimensions such that the flanges tightly fit in the sulcus and, secondly, designs with hooks, barbs or other mechanical adaptations which ensure firm positioning and connection of the haptics to the sulcus.

A third exemplary embodiment of an intraocular lens shown in FIG. 3 has three flat Ω-shaped spring-like haptics equally spaced around the rim of the optics.

A fourth exemplary embodiment of an intraocular lens shown in FIG. 4 has at least four flat Ω-shaped spring-like haptics equally spaced around the rim of the optics. The optics are preferably of a rotational symmetrical multifocal or monofocal design.

FIG. 1 shows details of a single flat Ω-shaped spring-like haptic with rim 1 which touches the capsular bag or the sulcus, depending on the positioning in the eye; the section of the spring-like structure from which most of the spring function originates 2; the opening in the spring 3 which flattens at compression; and the attachment component 4 which attaches the spring-like structure to the optics 5, in this exemplary embodiment, likely rotational symmetrical optics which will not shift relative to the optical axis at contraction of ciliary muscle or sulcus.

FIG. 2 shows an intraocular lens with flat Ω-shaped spring-like haptic in an exemplary embodiment with one such flat Ω-shaped spring-like haptic 6 opposite one sturdy, non-spring-like haptic 7. For details of the flat Ω-shaped spring-like haptic, refer to the description hereinabove regarding FIG. 1. At the opposite side of the flat Ω-shaped spring-like haptic 6, a sturdy non-spring-like haptic 7 is connected to the optics with an attachment component 4. The optics 10, 11 in this particular example is a progressive optics. At contraction of the ciliary muscle or sulcus (not illustrated), the rim 1 is compressed, closing the opening in the spring 3 and thereby shifting the optics perpendicular to the optical axis exposing the center of the optics to a sector of higher dioptre power 11, the degree of optical power denoted by the “+” signs.

FIG. 3 shows another exemplary embodiment of an intraocular lens in which three flat Ω-shaped spring-like haptics are arranged around the optics. An explanation of the components is disclosed hereinabove.

FIG. 4 shows yet another exemplary embodiment of an intraocular lens in which four flat Ω-shaped spring-like haptics form a circular spring-like ring around the optics. An explanation of the components is disclosed hereinabove.

All patents, patent applications and publications referred to herein are incorporated by reference in their entirety.

1. An intraocular lens, comprising:
a) a central optical element having optical surfaces which are smooth over their full areas; and
b) at least two haptics positioned in a plane perpendicular to the optical axis of the eye, wherein at least one of the haptics has a substantially Ω-shaped structure adapted to be compressed in a direction perpendicular to the optical axis.

2. The intraocular lens of claim 1, wherein the lens comprises a flexible Ω-shaped haptic arranged opposite a rigid Ω-shaped haptic.

3. The intraocular lens of claim 2, wherein the lens has progressive optical properties.

4. The intraocular lens of claim 3, wherein the optical strength of the lens increases in the direction between the two haptics.

5. The intraocular lens of claim 1, wherein the optics are designed such that relaxation of a structure of the eye results in emmetropic vision.

6. The intraocular lens of claim 5, wherein the optics are designed such that constriction of a structure in the eye results in accommodation.

7. The intraocular lens of claim 1, wherein the construction has at least two flexible Ω-shaped haptics.

8. The intraocular lens of claim 1, wherein the lens is implanted in the capsular bag of the eye.

9. The intraocular lens of claim 1, wherein the lens is positioned in the sulcus of the eye.

10. The intraocular lens of claim 1, wherein the haptics are made from the same material as the lens.
